(19) United States
(12) Patent Application Publication
(10) Pub. No.: US 2023/0218542 A1
(43) Pub. Date: Jul. 13, 2023

(54) MEDICAMENT FOR TREATING HYPERTENSION

(71) Applicant: Shumin LIU, Harbin (CN)

(72) Inventors: Shumin LIU, Harbin (CN); Donghua YU, Harbin (CN); Jinchuan ZHAO, Harbin (CN); Xin GAO, Harbin (CN); Nan ZHANG, Harbin (CN); Peng XU, Harbin (CN); Yang NAN, Harbin (CN)

(73) Assignee: Shumin LIU, Harbin (CN)

(21) Appl. No.: 17/970,577

(22) Filed: Oct. 21, 2022

(30) Foreign Application Priority Data
Jan. 12, 2022 (CN) 202210033465.1

(51) Int. Cl. A61K 31/05; A61K 31/4965; A61K 9/16; A61K 9/00

(52) U.S. Cl. CPC A61K 31/05; A61K 9/16; A61K 9/0019; A61K 9/0056; A61K 31/4965

(57) ABSTRACT

A medicament for treating hypertension is provided. The medicament has an active ingredient including cannabidiol or a pharmaceutically acceptable salt or ester thereof, and ligustrazine or a pharmaceutically acceptable salt or ester thereof is added to the active ingredient of the medicament. The medicament provided by the present invention has a good therapeutic effect on hypertension and can achieve a significant antihypertensive effect after being administered for a short time. The medicament for treating hypertension provided by the present invention has the advantages of low cost, low toxicity, stable chemical components, and continuous and stable antihypertensive effect.

### CROSS REFERENCE TO THE RELATED APPLICATIONS

This application is based upon and claims priority to Chinese Patent Application No. 202210033465.1, filed on Jan. 12, 2022, the entire contents of which are incorporated herein by reference.

### TECHNICAL FIELD

The present invention belongs to the technical field of pharmaceuticals, and particularly relates to a medicament for treating hypertension.

### BACKGROUND

Hypertension is the most common chronic disease and the most main risk factor for cardiovascular and cerebrovascular diseases. The main causative factors of hypertension are as follows: sex and age: researches find that the probability of women suffering from hypertension is much higher than that of men, and hypertension mostly occurs in the elderly population, which is one of the important diseases that threaten the life safety of the elderly; poor living and eating habits: for example, irregular life, often staying up late, daily food containing excessive salt and fat, heavy drinking or smoking, etc.; and greater living and working pressure: the increasing progress of society brings more cruel competition, so that the pressure in people is also increasing in this environment. Under the above-mentioned several factors, the number of patients with hypertension is increasing. At present, there are many types of medicaments for treating hypertension, but they often have the problems of slow efficacy, high cost and the like, so that there is a need to develop a medicament for treating hypertension that can quickly reduce blood pressure and have low cost.

### SUMMARY

Aiming at the above-mentioned defects in the prior art, the present invention provides a medicament for treating hypertension.

In order to achieve the above-mentioned objective, the present invention provides the following technical solutions.

The present invention provides a medicament for treating hypertension, wherein the medicament has an active ingredient comprising cannabidiol or a pharmaceutically acceptable salt or ester thereof.

Furthermore, the medicament has an active ingredient further comprising ligustrazine or a pharmaceutically acceptable salt or ester thereof.

As a preferred technical solution of the present invention, a mass ratio of the cannabidiol to the ligustrazine is 1:0.5 to 1:2.

Further preferably, the mass ratio of the cannabidiol to the ligustrazine is 1:1.

Furthermore, the medicament further comprises one or more pharmaceutically acceptable excipients.

Furthermore, a dosage form of the medicament comprises an oral liquid, a tablet, a capsule, a granule, a pill or an injection.

The present invention is based on modern medical theory, combines with the concept of compatibility in traditional Chinese medicine, develops the characteristics of combination of traditional Chinese medicine and western medicine, and selects the medicament monomers with low toxicity and complementary gains for compatibility, which have significant and continuous antihypertensive effect on specific animal models.

Specifically, the medicaments of the present invention are described below.

(1) The main medicament: cannabidiol (CBD), a pure natural component extracted from cannabis plants, has a molecular formula of C21H30O2. Because of its good nerve sedation and anti-inflammatory effects, and no addiction and significant toxicity, the medicament is widely used for treating various diseases such as epilepsy, anxiety, tumor, arthritis and the like. For the treatment of hypertension, only a small number of in vitro experiments have shown that CBD has the effect of dilating arterial vessels at present, and no literatures at home and abroad have reported that CBD can be independently used in clinical or in vivo trials for treating hypertension.

(2) The compatible medicament: ligustrazine (TMP), the main active ingredient of traditional Chinese medicine ligusticum chuanxiong hort, has a molecular formula of C8H12N2. It has effects of dilating peripheral vessels, increasing coronary flow, improving microcirculation and resisting thrombosis, and it can be used for treating occlusive vascular disease, cerebral thrombosis, vasculitis, coronary heart disease, angina pectoris, etc.

Compared with the prior art, the present invention has the following beneficial effects:
The medicament for treating hypertension provided by the present invention has a good therapeutic effect on hypertension, and can achieve a significant antihypertensive effect after being administered for a short time;
the medicament for treating hypertension provided by the present invention has the advantages of low cost, low toxicity, stable chemical components and continuous and stable antihypertensive effect.

### BRIEF DESCRIPTION OF THE DRAWINGS

In order to more clearly illustrate the technical solutions in the embodiments of the present invention or in the prior art, the drawings required to be used in the description of the embodiments are briefly introduced below. It is obvious that the drawings in the description below are some embodiments of the present invention, and those of ordinary skilled in the art can obtain other drawings according to the drawings provided herein without creative efforts.

FIG. 1 is a graph showing the distribution of SBP measured in control rats.

FIG. 2 is a graph showing the change trend of SBP of rats in each group during the administration period in Effect Verification 1.

FIG. 3 is a graph showing the change trend of DBP of rats in each group during the administration period in Effect Verification 1.

FIGS. 4A-4B are graphs showing the results of blood pressure measurement of rats in each group before and after the administration in Effect Verification 1, wherein FIG. 4A is the results of SBP measurement in each group before and after the administration, and FIG. 4B is the results of DBP measurement in each group before and after the administration.

FIG. 5 shows the results of combination index (CI) analysis in the combination of CBD + TMP in Effect Verification 1.

FIGS. 6A-6B are graphs showing the change trend of blood pressure of rats in each group during the administration period in Effect Verification 2, wherein FIG. 6A is the change trend of SBP, and FIG. 6B is the change trend of DBP.

FIGS. 7A-7B are graphs showing the change trend of blood pressure of rats in each group during the administration period in Effect Verification 3, wherein FIG. 7A is the change trend of SBP, and FIG. 7B is the change trend of DBP.

### DETAILED DESCRIPTION OF THE EMBODIMENTS

Various exemplary embodiments of the present invention have been described in detail, and the detailed description should not be considered as limiting the present invention, but should be understood as a more detailed description of certain aspects, features and embodiments of the present invention. It should be understood that the terms described in the present invention are for the purpose of describing particular embodiments only and are not intended to limiting the present invention.

In addition, for numerical ranges in the present invention, it should be understood that each median value between the upper and lower limits of the range is also specifically disclosed. Every smaller range between any stated value or median value within a stated range and any other stated value or median value within the stated range is included within the present invention. The upper and lower limits of these smaller ranges may independently be included or excluded in the range.

Unless defined otherwise, all technical and scientific terms used herein have the same meaning as commonly understood by those of ordinary skill in the art described in the present invention. Although only preferred methods and materials are described in the present invention, any methods and materials similar or equivalent to those described herein can also be used in the implementation or testing of the present invention. All literatures mentioned in the specification are incorporated herein by reference to disclose and describe the methods and/or materials associated with the literatures. In case of conflict with any incorporated literature, the content of the specification shall prevail.

It will be apparent to those skilled in the art that various modifications and changes can be made in the specific embodiments of the present invention without departing from the scope or spirit of the present invention. Other embodiments obtained from the specification of the present invention will be apparent to those skilled in the art. The specification and embodiments of the present invention are exemplary only.

As used herein, the terms “comprising”, “including”, “having”, “containing” and the like are open-ended, which mean including but not limited to.

Medicaments, reagents and experimental animals used in the following examples are shown as follows:
cannabidiol (CBD): Yunnan Hansu Biotechnology Co., Ltd., specification: ultra-pure powder (CBD ≥ 99.5%, THC is undetectable), Batch No.: 20200604;
ligustrazine phosphate (TMP): specification: 50 mg/tablet, Guangdong Livzon Group Limin Pharmaceutical Factory, Batch No.: H44024348;
L-NAME (eNOS inhibitor): Shanghai Beyotime Biological Tech. Co., Ltd., model: S0006;
rat common feed and 8% high-salt feed: Guangdong Medical Laboratory Animal Center, Batch No.: 44200300022971;
experimental animals: SPF grade, male 6-week-old SD rats, body weight: (180 ± 20) g, purchased from Liaoning Changsheng Biotechnology Co., Ltd., No.: 2021062863, use license No.: SYXK (Black) 2018-007;
instruments used in examples: ALC-NIBP noninvasive blood pressure measuring instrument (Shanghai Alcott Biotechnology Co., Ltd.); AL204 electronic balance (Mettler-Toledo Instrument Co., Ltd.).

The medicaments provided by the present invention and the raw materials and reagents used in the extraction process thereof can be purchased from the market.

### Example 1

CBD was dissolved in edible oil, and ultrasonic oscillation was performed until the mixed solution was completely dissolved to obtain a CBD solution. The solution was stored at 4° C. away from light for later use;

TMP was dissolved in distilled water, and the shaking was performed until the mixed solution was completely dissolved to obtain a TMP solution, which was prepared freshly when needed.

### Example 2

Cistanche deserticola aqueous extract was prepared as follows: 10 g of cistanche deserticola was added to 150 mL of water, the solution was heated to 90° C. and incubated for 5 h, followed by filtration. The resulting filtrate was concentrated at 50° C. under reduced pressure, and then dried at 70° C. to obtain the cistanche deserticola aqueous extract.

The resulting cistanche deserticola aqueous extract was dissolved in water for later use.

### Example 3

Cistanche deserticola acetic acid solution extract was prepared as follows: 10 g of cistanche deserticola was added to 150 mL of acetic acid solution with a concentration of 10 wt% for heating and reflux extraction, the heating temperature was 90° C. and the extraction time was 5 h, followed by filtration. The resulting filtrate was concentrated at 50° C. under reduced pressure, and then dried at 70° C. to obtain the cistanche deserticola acetic acid solution extract.

The resulting cistanche deserticola acetic acid solution extract was dissolved in water, and then uniformly dispersed by ultrasonic for later use.

After the medicaments and composition thereof of the present invention are combined with pharmaceutically acceptable excipients, they are prepared into a suitable dosage form according to the conventional formulation method in the art. The pharmaceutical composition may be an oral liquid, a tablet, a capsule, a pill, a granule and an injection.

The present invention refers to the dosage in Chinese Pharmacopoeia (2020 edition) and related literatures, takes CBD 30-160 mg/kg and TMP 30-80 mg/kg as dosage ranges for rats, and adopts L-NAME medicament modeling method and high-salt modeling method to prepare hypertensive model rats. The change trends of systolic blood pressure and diastolic blood pressure before and after the administration are determined by noninvasive measurement of tail artery pressure, so as to detect the using effects of CBD and its compatibility with TMP in different ratios in the treatment of hypertension.

### Effect Verification 1

Therapeutic Effect of the Medicament Provided in Example 1 of the Present Invention on L-NAME-Induced Hypertensive Rats

### 1. Blood Pressure Measurement and Hypertension Range Determination

The tail artery pressure, including systolic blood pressure (SBP) and diastolic blood pressure (DBP), was measured in rats in the resting state at the same time using an ALC-NIBP noninvasive blood pressure measuring instrument. One hundred and fifty-two 6-week-old male SD rats were taken and subjected to adaptive feeding for one week at room temperature of 18-26° C. with the relative humidity of 40%-70% and illumination by artificial lighting from 9:00 to 21:00 every day. After one week, blood pressure in all of them was measured, the results were charted using Excel 2010, and the experimental data were analyzed using SPSS 20.0, where P < 0.05 indicated statistical significance and the measurement data were expressed as x̅ ± s. The measured SBP results are shown in FIG. 1, which are in accordance with normal distribution (Shapiro-Wilk normality test, P = 0.605), indicating that the rat strain, the feeding environment and the instrument are all in a stable state. According to the commonly used grading criteria for hypertension, SBP 140 mmHg is the critical value of hypertension, and every 20 mmHg is one grade. Every 10 mmHg of DBP is one grade. Therefore, SBP > 140 mmHg is set as mild hypertension and SBP > 160 mmHg is set as moderate hypertension; optionally, DBP increased by more than 10 mmHg is mild hypertension and DBP increased by more than 20 mmHg is moderate hypertension.

### 2. Model Preparation and Administration

Rats with the blood pressure in the normal range measured in the step 1 above and the weight of 200 ± 20 g were randomly taken and grouped into 7 groups: a control group (K) containing 8 rats, a model control group (M), a ligustrazine administration group (TMP, 60 mg/kg), a cannabidiol administration group (CBD, 60 mg/kg), and combined administration groups of TMP and CBD (divided into three groups in different ratios of 2:1 (XA), 1:1 (XB) and 1:2 (XC), TMP = 60 mg/kg, and CBD was 30 mg/kg, 60 mg/kg and 120 mg/kg, respectively). 10 rats were contained in each remaining group. Except for group K, 12 mg/kg L-NAME was administered intragastrically to the rest at 9:00 every day to manufacture a hypertension model, and distilled water was administered in an equal amount to K group. By the fourth week, blood pressure was measured in each group, and after individuals with SBP < 140 mmHg were excluded from the model, each group was administered for three weeks, and drug vehicles were administered in an equal amount to groups K and M. Blood pressure was measured once a week during the administration period. (The animal model was fasted for two hours before the administration, and the medicament was bathed to room temperature. TMP solution at 1 mL/100 g was firstly administered intragastrically to the combined administration groups of TMP and CBD, and after 1 h interval, CBD solution at the corresponding half volume was then administered intragastrically.)

### 3. Evaluation of Medicament Efficacy

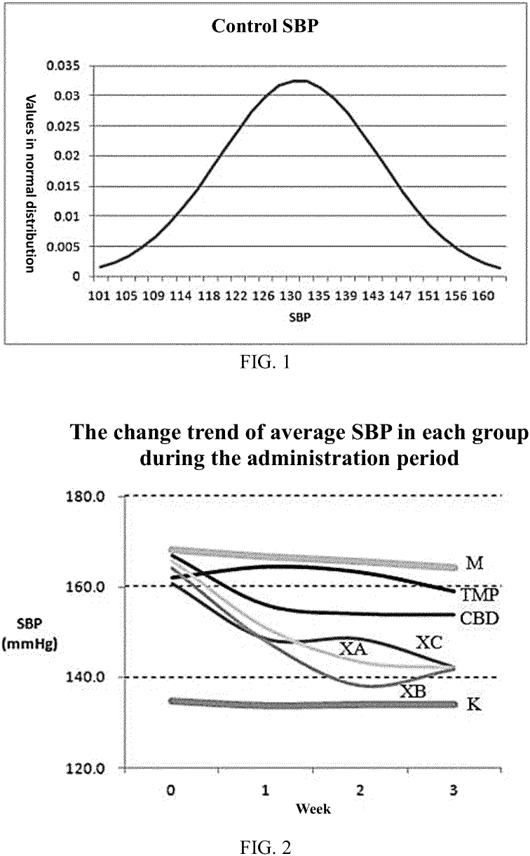

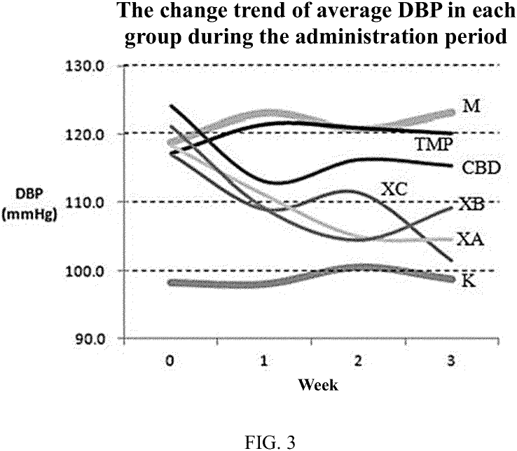

One-way ANOVA analysis (Duncan) was used for pairwise comparisons among groups, and paired-sample t-test was used for before-and-after comparisons within groups. The change trend of SBP of rats in each group is shown in FIG. 2 and the change trend of DBP of rats in each group is shown in FIG. 3, and the abscissa “0” represents the fourth week of molding, i.e., the beginning of the administration period.

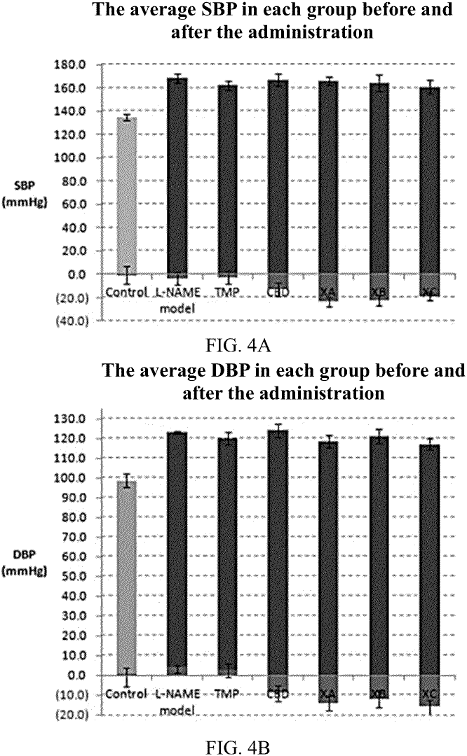

The results of blood pressure measurement of rats in each group before and after the administration are shown in FIGS. 4A-4B, wherein FIG. 4A is the results of SBP measurement in each group before and after the administration, and FIG. 4B is the results of DBP measurement in each group before and after the administration; the positive coordinate values represent the SBP/DBP in each group before the administration, i.e., the fourth week of modeling, and each group has significant difference compared with the group K (P < 0.001); the negative coordinates represent the absolute decrease of SBP/DBP in each group at the third week of administration, i.e., after the end of the administration.

As shown in FIGS. 2, 3 and 4A-4B, the SBP in each model group was greater than 160 mmHg at the fourth week of molding, which had significant difference compared with the group K, and the increase in SBP was greater than 30 mmHg and the increase in DBP was greater than 20 mmHg relative to the pre-modeling period, which reached the moderate hypertension criteria; compared with the group M, the other model groups had no significant difference. Therefore, the model preparation was successful.

After the end of the administration, the comparison of SBP in each administration group with that in the group M showed that the TMP group had no significant difference, the CBD group had significant difference (P < 0.05), and the composition groups had highly significant difference (P < 0.01). Both CBD group and composition groups decreased by more than 10 mmHg. In particular, SBP in XB group had dropped to the lowest point to the normal range at the second week of administration, and a maximum decrease in the average SBP was up to 25.9 mmHg; in addition, comparisons among groups before and after the second and third weeks of administration showed that there was no significant difference, indicating that the blood pressure in each group tended to be stable at the second week of administration, so that the medicament achieved the best efficacy.

The results above show that both the CBD and the CBD + TMP combination have significant antihypertensive effect, so that the systolic blood pressure and the diastolic blood pressure of the hypertensive rat model can be significantly and synchronously decreased, and the antihypertensive effect of the composition is more significant, particularly, the composition has the fastest effect and the best efficacy when being prepared in a ratio of 1:1; while TMP used independently has no significant antihypertensive effect, suggesting that CBD exerts a major antihypertensive effect.

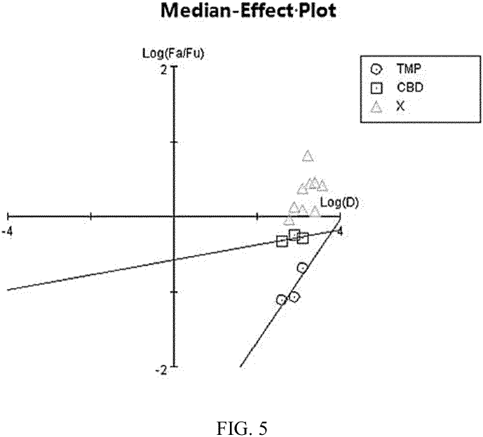

Further investigation of the combination index in the combination of CBD + TMP was preformed, and the results are shown in Table 1 and FIG. 5.

TABLE 1

| Administration | Effect | CI |
|---|---|---|
| One week in TMP | 0.0716 | / |
| Two weeks in TMP | 0.0780 | / |
| Three weeks in TMP | 0.1722 | / |
| One week in CBD | 0.3222 | / |
| Two weeks in CBD | 0.3628 | / |
| Three weeks in CBD | 0.3418 | / |
| One week in XA | 0.4826 | 0.04175 |
| Two weeks in XA | 0.7063 | 0.02582 |
| Three weeks in XA | 0.7363 | 0.03228 |
| One week in XB | 0.572 | 0.02646 |
| Two weeks in XB | 0.8695 | 0.00745 |
| Three weeks in XB | 0.7458 | 0.03039 |
| One week in XC | 0.5533 | 0.02917 |
| Two weeks in XC | 0.5417 | 0.06196 |
| Three weeks in XC | 0.7273 | 0.03414 |

Note: Effect = (SBP in model group - SBP in administration group)/(SBP in model group - SBP in control group).

As can be seen from Table 1, the CI values in the combination administration groups are less than 0.1 at different periods, indicating that TMP and CBD have significant combined antihypertensive effect.

### Effect Verification 2

Therapeutic Effect of the Medicament Provided in Example 1 of the Present Invention on High-Salt Diet-Induced Hypertensive Rats

### 1. Blood Pressure Measurement and Hypertension Range Determination

It is same as step 1 in “Effect Verification 1”.

### 2. Model Preparation and Administration

Rats with the blood pressure in the normal range measured in the step 1 above were randomly taken and grouped into 3 groups: a control group (K) containing 8 rats, a model control group (M) and a model administration group (D, CBD = 80 mg/kg, TMP = 80 mg/kg). 10 rats were contained in each remaining group. Groups M and D were administered 8% high-salt feed for six weeks to manufacture a hypertension model, and group K was administered common feed. By the sixth week, blood pressure was measured in each group, and after individuals with SBP < 140 mmHg were excluded from the model, the administration group was administered for three weeks, and drug vehicles were administered in an equal amount to groups K and M with administration of common feed. Blood pressure was measured once a week during the administration period. (The animal model was fasted for two hours before the administration, and the medicament was bathed to room temperature. TMP solution at 1 mL/100 g was firstly administered intragastrically to the combined administration groups of TMP and CBD, and after 1 h interval, CBD solution at the corresponding half volume was then administered intragastrically.)

### 3. Evaluation of Medicament Efficacy

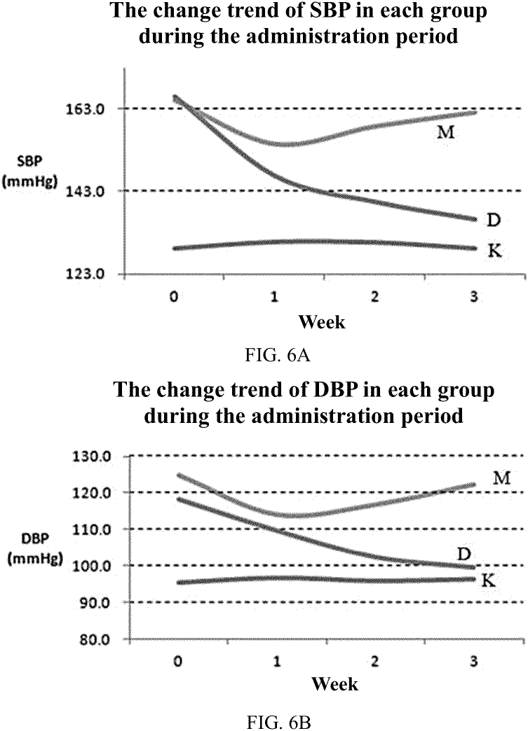

One-way ANOVA analysis (Duncan) was used for comparisons among groups, and paired-sample t-test was used for before-and-after comparisons within groups. The change trend of blood pressure of rats in each group is shown in FIGS. 6A-6B, wherein FIG. 6A is the change trend of SBP and FIG. 6B is the change trend of DBP, and the abscissa “0” represents the sixth week of molding, i.e., the beginning of the administration period.

As shown in FIGS. 6A-6B, the SBP in each model group was greater than 160 mmHg at the sixth week of molding, which had significant difference compared with the group K, and the increase in SBP was greater than 30 mmHg and the increase in DBP was greater than 20 mmHg relative to the pre-modeling period, which reached the moderate hypertension criteria; compared with the group M, the group D had no significant difference. Therefore, the model preparation was successful.

After the end of the administration, the SBP and DBP in each group were compared with those of the group before the administration, indicating that the groups K and M had no significant difference, and the group D had significant difference. The SBP in the group D had decreased to the normal range with a decrease of 29.75 ± 7.13 mmHg, and the DBP had decreased by 18.63 ± 5.40 mmHg; after the end of the administration, SBP and DBP in the group D had significant difference compared with those in the group M.

The results above show that the combination of CBD and TMP in a ratio of 1:1 has significant antihypertensive effect, so that the systolic blood pressure and the diastolic blood pressure of the hypertensive rat model can be significantly and synchronously decreased to the normal level of the blood pressure in a two- to three-week treatment course.

### Effect Verification 3

Therapeutic Effect of the Medicament Provided in Examples 1 to 3 of the Present Invention on High-Salt Diet-Induced Hypertensive Rats

### 1. Blood Pressure Measurement and Hypertension Range Determination

It is same as step 1 in “Effect Verification 1”.

### 2. Model Preparation and Administration

Rats with the blood pressure in the normal range measured in the step 1 above were randomly taken and grouped into 5 groups: a control group (K) containing 8 rats, a model control group (M), a model administration group XA (CBD = 80 mg/kg, TMP = 80 mg/kg), a model administration group XB (CBD = 80 mg/kg, TMP = 80 mg/kg, cistanche deserticola aqueous extract = 80 mg/kg) and a model administration group XC (CBD = 80 mg/kg, TMP = 80 mg/kg, cistanche deserticola acetic acid solution extract = 80 mg/kg). 10 rats were contained in each remaining group. Except for group K, the other 4 groups were administered 8% high-salt feed for six weeks to manufacture a hypertension model, and group K was administered common feed. By the sixth week, blood pressure was measured in each group, and after individuals with SBP < 140 mmHg were excluded from the model, the administration group was administered for three weeks, and drug vehicles were administered in an equal amount to groups K and M with administration of common feed. Blood pressure was measured once a week during the administration period. (The animal model was fasted for two hours before the administration, and the medicament was bathed to room temperature. An administration method in the group XA was as follows: TMP solution at 1 mL/100 g was firstly administered intragastrically, and after 1 h interval, CBD solution at the corresponding half volume was then administered intragastrically; and administration methods in the groups XB/XC were as follows: TMP solution at 1 mL/100 g was firstly administered intragastrically, and after 1 h interval, CBD solution at the corresponding half volume and the solution of cistanche deserticola aqueous extract/cistanche deserticola acetic acid solution extract at the corresponding half volume were then administered intragastrically.)

### 3. Evaluation of Medicament Efficacy

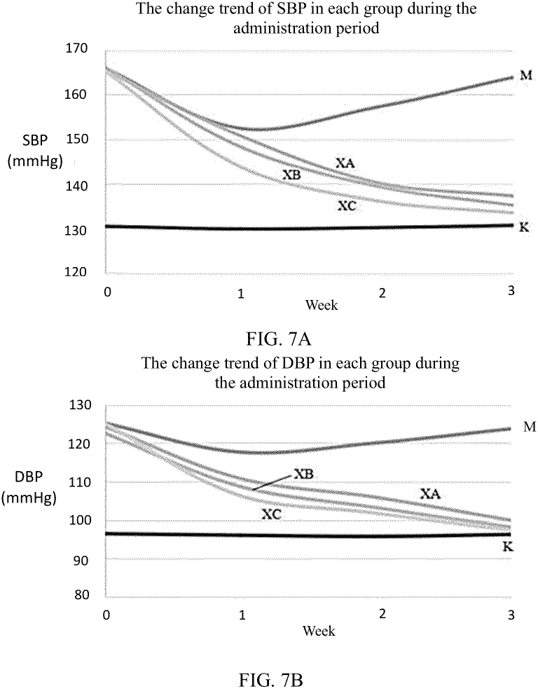

One-way ANOVA analysis (Duncan) was used for comparisons among groups, and paired-sample t-test was used for before-and-after comparisons within groups. The change trend of blood pressure of rats in each group is shown in FIGS. 7A-7B, wherein FIG. 7A is the change trend of SBP and FIG. 7B is the change trend of DBP, and the abscissa “0” represents the sixth week of molding, i.e., the beginning of the administration period.

As shown in FIGS. 7A-7B, the SBP in each model group was greater than 160 mmHg at the sixth week of molding, which had significant difference compared with the group K, and the increase in SBP was greater than 30 mmHg and the increase in DBP was greater than 20 mmHg relative to the pre-modeling period, which reached the moderate hypertension criteria; compared with the group M, the groups XA, XB and XC had no significant difference. Therefore, the model preparation was successful.

After the end of the administration, the SBP and DBP in each group were compared with those of the group before the administration, indicating that the groups K and M had no significant difference, and the groups XA, XB and XC had significant difference, wherein the group XC had the best efficacy on hypertension. After the end of the administration, SBP and DBP in the group D had significant difference compared with those in the group M.

The results above show that the addition of a certain amount of the cistanche deserticola aqueous extract or the cistanche deserticola acetic acid solution extract to the combination of CBD and TMP in a ratio of 1:1 can further enhance the antihypertensive effect thereof on hypertension.

The above description is only the preferred embodiment of the present invention, and the protection scope of the present invention is not limited thereto. According to the technical solution of the present invention and the inventive concept thereof, equivalents or changes made by those skilled in the art within the technical scope of the present disclosure shall fall within the protection scope of the present invention.

What is claimed is:

1. A medicament for treating hypertension, wherein the medicament has an active ingredient comprises cannabidiol or a pharmaceutically acceptable salt of the cannabidiol or a pharmaceutically acceptable ester of the cannabidiol.

2. The medicament for treating the hypertension according to claim 1, wherein the active ingredient further comprises ligustrazine or a pharmaceutically acceptable salt of the ligustrazine or a pharmaceutically acceptable ester of the ligustrazine.

3. The medicament for treating the hypertension according to claim 2, wherein a mass ratio of the cannabidiol to the ligustrazine is 1:0.5 to 1:2.

4. The medicament for treating the hypertension according to claim 3, wherein the mass ratio of the cannabidiol to the ligustrazine is 1:1.

5. The medicament for treating the hypertension according to claim 1, further comprising one or more pharmaceutically acceptable excipients.

6. The medicament for treating the hypertension according to claim 5, wherein a dosage form of the medicament comprises an oral liquid, a tablet, a capsule, a granule, a pill, or an injection.

7. The medicament for treating the hypertension according to claim 2, further comprising one or more pharmaceutically acceptable excipients.

8. The medicament for treating the hypertension according to claim 7, wherein a dosage form of the medicament comprises an oral liquid, a tablet, a capsule, a granule, a pill or an injection.
